(19) United States
(12) Patent Application Publication
(10) Pub. No.: US 2018/0344953 A1
(43) Pub. Date: Dec. 6, 2018

(54) INHALATION SYSTEM

(71) Applicant: R. Cegla GmbH & Co. KG, Montabaur (DE)

(72) Inventor: Ulrich Hartmann Cegla, Montabaur (DE)

(21) Appl. No.: 15/997,274

(22) Filed: Jun. 4, 2018

(30) Foreign Application Priority Data
Jun. 2, 2017 (DE) EP 17 174 196.0

(51) Int. Cl. A61M 15/00

(52) U.S. Cl. CPC A61M 15/002; A61M 2206/20; A61M 2206/12; A61M 15/009

(57) ABSTRACT

In an inhalation system (1) for inhalation of a dosing aerosol (6′), comprising:
a dosing device (6) which delivers the aerosol (6′)
a container (2) in which one or more chambers (4, 5) separated from one another and connected together during inhalation by means of one or more valves (8) are provided,
with an inlet opening (7) emerging into the first chamber (4), into which opening the dosing device (6) is inserted,
with the second chamber (5) having a first passage opening (9) which interacts with the respiratory openings of a human being (20),
with the second chamber (5) having a second passage opening (10) that is connected to the surrounding air (15) and through which a positive pressure in the second chamber (5) can escape into the surrounding air (15) during exhalation, a simple and hygienic application of the inhalation arrangement (1) should be guaranteed and the aerosol flow should have a linear and rotating motion induced in it during the inhalation procedure.

This is achieved in that
the valve (8) is formed from an elastically deformable material, preferably silicone, that the valve (8) has a ring-shaped circumferential collar (21) that is fixed to the container (2),
at least one slot-shaped notch (22) is worked into the valve (8) which is configured as a spiral running from inside to outside,
that during inhalation, the valve (8) acts as a barrier to the flow of the aerosol (6′) flowing through it, as a result of which a linear motion and a spiral-shaped flow direction (11) along and about the longitudinal axis (3) of the container (2) is imposed on the aerosol (6′), channelling it into a three-dimensional spiral

### REFERENCE TO PENDING PRIOR PATENT APPLICATION

This patent application claims benefit of European Patent Application No. 17 174 196.0, filed Jun. 2, 2017, which patent application is hereby incorporated herein by reference.

### FIELD OF THE INVENTION

The present invention relates to an inhalation system for inhaling a dosed aerosol in accordance with the pre-characterising clauses of patent claim 1.

### BACKGROUND OF THE INVENTION

An inhalation arrangement of this kind has been disclosed, for example, in EP 2 678 060 B1. Inhalation arrangements of this kind consist of a container with two chambers connected together by means of a valve. In the aforementioned state of the art, the valve is configured as a diaphragm into which two slits running at right angles to one another are worked, the slits closing during the exhalation procedure; on the other hand, during inhalation the cross-slits formed in this way are opened with the result that the aerosol input into the first chamber can flow from there into the second chamber. The aerosol is input by means of a dosing device that is arranged on an inlet opening which emerges into the first chamber.

As a result of the increase in volume between the dosing device and the first chamber, the aerosol is distributed evenly within the first chamber and can be inhaled by a patient using a normal inhalation procedure. During the inhalation procedure, a negative pressure is created in the second chamber which opens the valve between the first and second chambers with the result that the aerosol flows in from the first into the second, and from there through a first passage opening into a mouthpiece which is in contact with the mouth of a patient, for example.

During the exhalation procedure, the second chamber is to be separated from the first chamber by closing of the valve, with the result that no respiratory air can flow in from the second chamber to the aerosol-filled first chamber. Rather, the air exhaled by the patient should escape from the second chamber into the surrounding air by means of a second passage opening.

Although handling of the inhalation arrangement is simple and can be accomplished hygienically because the individual components forming the inhalation arrangement can be sterilised, it has proven to be a disadvantage in this state of the art that there is an almost linear, cylindrical air flow during the inhalation procedure because the cross-slits worked into the diaphragm are at right angles to one another, with the effect that these scarcely exert any influence on the flow of the aerosol flowing from the first to the second chamber. As a result, the mixing between the aerosol drawn into the second chamber and the air present there from the surrounding air is slight or even non-existent, because to a large extent the diaphragm forms a cylindrical aerosol flow which scarcely mixes at all with the air drawn in from the surrounding air into the second chamber. As a result, highly concentrated aerosol enters the patient's mouth, and has a low effect in relation to the inhaled quantity of aerosol because there is no optimum distribution of the aerosol in the respiratory passages and it frequently fails to reach the bronchi or lungs in adequate quantity because the aerosol has already been deposited on the mucous membranes of the mouth or throat.

### SUMMARY OF THE INVENTION

The task of the present invention is therefore to create an inhalation arrangement of the aforementioned kind in such a way that, firstly, simple and hygienic use of the inhalation arrangement is guarantee and, secondly, a linear and rotating motion is induced in the aerosol flow during the inhalation procedure which chiefly moves along and about the longitudinal axis of the container of the inhalation arrangement, as a result of which a rotational motion of the aerosol created in this way enters the mouth, throat and lung area of the patient so that the aerosol also transported is distributed as widely and evenly as possible to the area of the patient which is to be treated.

These tasks are accomplished by the features in the pre-characterising clause of patent claim 1.

Further advantageous configurations of the invention are disclosed in the subordinated claims.

Due to the fact that the valve is formed from a flexibly deformable material, preferably silicone, that the valve has a ring-shaped circumferential collar that is fixed to the container, that a slot-shaped notch is worked into the valve which is configured as a spiral reducing in diameter from the outside to the inside, and that during inhalation, the notches of the valve present a barrier to the flow of the aerosol flowing through them as a result of which a linear and rotating motion is induced in the aerosol along and about the longitudinal axis of the container, the effect achieved is that the aerosol drawn into the second chamber through the configuration of the valve undergoes a linear forwards motion and rotation about the longitudinal axis of the container, which in addition presents different diameters. The valve, namely, consists of a spiral-shaped structure running from the inside to the outside which acts as a barrier to the flow of the inhaled aerosol during the inhalation procedure and consequently deflects this aerosol in the corresponding direction as a result of which a three-dimensional flow contour is created in the aerosol during each inhalation cycle. Such an aerosol structure is deposited at entirely different positions in the mouth, throat and lung area of the patient during the inhalation procedure, as a result of which an evenly penetrating treatment by aerosol is ensured for all areas of the human being which require application of this medicine.

Configuration of the valve in a ring-shaped circumferential collar guarantees that the valve can be fixed in a precise position and orientation on the container. The container is configured in two parts, with the effect that the valve can be exchanged quickly so that it can be sterilised or cleaned, for example, in a hot water bath or a microwave.

The valve is manufactured from an elastically deformable material, preferably silicone, as a result of which, firstly, the preload force of the valve counteracts the positive pressure in the first chamber with the effect that the aerosol is exclusively transferred from the first chamber to the second chamber when the patient creates a negative pressure in the second chamber by inhalation as a result of which the valve is lifted in a spiral arrangement in conjunction with the positive pressure in the first chamber, and secondly, that the valve has a high level of shape stability in any desirable position which means it will not kink or snap through.

### BRIEF DESCRIPTION OF THE DRAWINGS

The drawing shows a sample embodiment of an inhalation arrangement configured in accordance with the present invention, the details of which are explained below. In the drawing,

FIG. 1 shows a side view of an inhalation system consisting of a two-piece container in which a valve is held, by means of which the container is divided into two chambers, of a dosing device which emerges into the first chamber of the container and in which there are two passage openings provided in the second chamber for inhalation and exhalation,

FIG. 2a shows the inhalation system in accordance with FIG. 1 in a magnified view during the inhalation procedure and

FIG. 2b shows the inhalation system in accordance with FIG. 1 during the exhalation procedure.

### DETAILED DESCRIPTION OF THE PREFERRED EMBODIMENTS

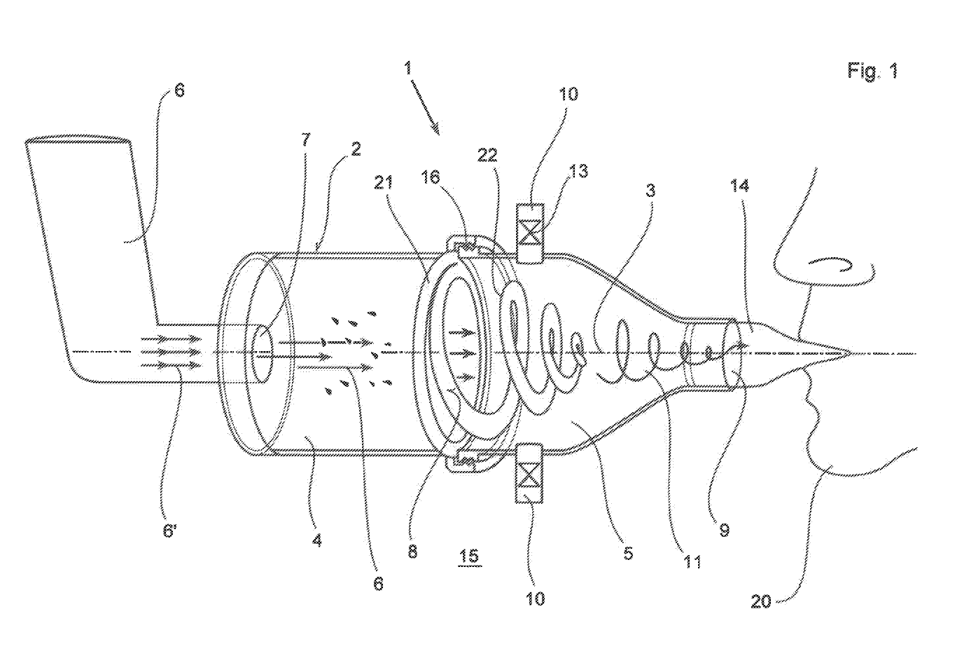

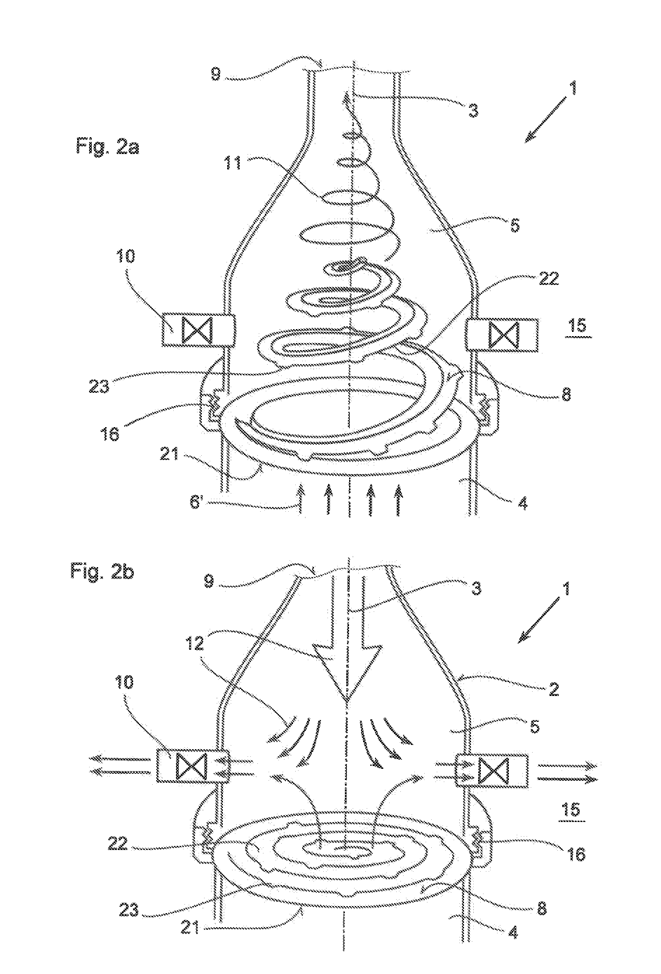

FIGS. 1 and 2a show an inhalation system 1 which serves the purpose of transporting an aerosol 6′ delivered by a dosing device 6 to a human being 20 for the treatment of respiratory passage illnesses. In particular, it is to be ensured that the dosing aerosol 6′ reaches the bronchial area of the patient 20 as evenly and optimally dosed as possible, and can be deposited evenly on the parts of the human being 20 which are to be treated.

The inhalation system 1 consists of a container 2 with a longitudinal axis with the reference number 3. The container 2 has two parts which are connected together but can be released. The transitional area between the two parts of the container 2 is defined as the parting plane into which a valve 8 explained in more detail below is inserted and locked in an orientated position. As a result, two chambers 4 and 5 are created inside the container 2 which are separated from one another by means of the valve 8, although they can communicate with one another when the valve 8 is opened. As soon as the valve 8 is closed, both chambers 4 and 5 are separated from one another in an airtight arrangement.

The first chamber 4 in the area of the longitudinal axis 3 of the container 2 has an inlet opening 7 worked into it, into which the dosing device 6 is inserted. Actuating the dosing device 6 causes the aerosol 6′ to enter the space formed by the first chamber 4 parallel to the longitudinal axis 3, and consequently it is distributed within the first chamber 4.

Two passage openings 9 and 10 are provided in the second chamber 5. The first passage opening 9 lies flush with the longitudinal axis 3 of the container 2 and can be provided with a mouthpiece 14, for example, which can be inserted into the mouth of the patient 20 for inhalation and exhalation. The passage openings 10 are located at the base of the spiral 21 and open during exhalation or close during inhalation.

FIG. 2a in particular shows that the valve 8 is formed from a ring-shaped circumferential collar 21 which is inserted between the two parts of the container 2 in the area of the thread 16, and is thus fixed in an orientated arrangement between the two parts of the container 2. The valve 8 is configured level and runs at right angles to the longitudinal axis 3 of the container 2.

Actuation of the dosing device 6 causes aerosol 6′ to enter the first chamber 4 and is distributed as evenly as possible therein. As a result, a positive pressure prevails in the first chamber 4. The valve 8 is manufactured from an elastically deformable material, preferably silicone. This material has a preload force which is sufficiently high for it to hold the positive pressure of the aerosol 6′ within the first chamber 4. Consequently, the valve 8 has an airtight closure and the aerosol 6′ within the first chamber 4 cannot escape from there into the second chamber 5. It is only when the patient 20 generates a negative pressure in the second chamber 5 by inhalation that the valve 8 opens, thereby allowing the aerosol 6′ to flow in from the first chamber 4 through the valve 8 into the second chamber 5.

The valve 8 has a spiral-shaped notch 22 reducing in diameter from the outside to the inside. Thus, as soon as the patient 20 inhales—which is referred to as the inhalation procedure—the valve 8 is lifted in a spiral shape. Consequently, in this condition, the valve 8 has a circumferential flow contour which takes on a three-dimensional shape. The three-dimensional structure of the valve 8 created in this way acts as a flow barrier to the aerosol 6′ that is drawn in, as a result of which a spiral-shaped rotation about the longitudinal axis 3 and simultaneously a linear advance movement is imposed on the aerosol 6′ which flows in the direction of the first passage opening 9 or is drawn in by the patient 20. The flow direction of the inhaled aerosol 6′ created in this way is referred to by number 11. All airflow is run parallel to the longitudinal axis 3 and about it, which means an aerosol flow 11 created in this way reaches the mouth, throat and lung area of the patient 20 with the effect that the aerosol 6′ flows can be evenly deposited on the areas of the patient 20 which are to be treated. As a result of the geometrical arrangement of the valve 8 with its spiral shaped-notch 22, and aerosol flow is created because a correspondingly sized diameter exists for each coil of the spiral-shaped notch 22.

The material of the valve 8 is manufactured from an elastically deformable material, preferably silicone, although nevertheless this material does possess a certain level of shape stability which means that the opened valve 8 does not kink out of shape. The valve 8 can, moreover, be removed from the container 2 so as to be cleaned in a hot water bath or a microwave. If the valve 8 undergoes wear as a result of intensive use, it can be exchanged or renewed in a straightforward procedure.

FIG. 2b shows the exhalation procedure of the patient 20 and its effect on the valve 8. The flow direction of the exhaled air is identified with the reference number 12 and flows through the mouthpiece 14 into the second chamber 5. It is essential to prevent the air exhaled by the patient 20 from entering the first chamber 4 and mixing with the aerosol 6′ that has been input there, thereby moistening it. As a result, it is necessary for the valve 8 to be closed airtight during the exhalation procedure of the patient 20. Consequently, the air exhaled by the patient 20 should escape from the second chamber 5 through the second passage opening 10 into the surrounding air 15. In this condition, the valve 8 closes both chambers 4 and 5 of from one another in an airtight arrangement.

In order to prevent the valve 8 from snapping through or undergoing deformation during the exhalation procedure by the patient 20, contact plates 23 are formed in the area of the notch 22 which are supported against the correspondingly adjacent band of the valve 8 in an overlapping arrangement from the inside to the outside. Consequently, the contact plates 23 produce an overlapping structure which counteracts the positive pressure in the second chamber 5 and thus achieves a stable shape and airtight structure for the valve 8.

1. An inhalation system (1) for inhalation of a dosing aerosol (6′), comprising:
a dosing device (6) which delivers the aerosol (6′)
a container (2) in which one or more chambers (4, 5) separated from one another and connected together during inhalation by means of one or more valves (8) are provided,
with an inlet opening (7) emerging into the first chamber (4), into which opening the dosing device (6) is inserted,
with the second chamber (5) having a first passage opening (9) which interacts with the respiratory openings of a human being (20),
with the second chamber (5) having a second passage opening (10) that is connected to the surrounding air (15) and through which a positive pressure in the second chamber (5) can escape into the surrounding air (15) during exhalation,
characterised in that,
the valve (8) is formed from an elastically deformable material, preferably silicone, that the valve (8) has a ring-shaped circumferential collar (21) that is fixed to the container (2),
at least one slot-shaped notch (22) is worked into the valve (8) which is configured as a spiral running from inside to outside,
that during inhalation, the valve (8) acts as a barrier to the flow of the aerosol (6′) flowing through it, as a result of which a linear motion and a spiral-shaped flow direction (11) along and about the longitudinal axis (3) of the container (2) is imposed on the aerosol (6′), channelling it into a three-dimensional spiral.

2. The inhalation arrangement in accordance with claim 1, characterised in that, one or more overlapping contact plates (23) are provided in the area of the notches (22) of the valve (8), by means of which plate(s) the valve (8) is closed with a stable shape and air-tight closure during exhalation.

3. The inhalation arrangement in accordance with claim 1, characterised in that, the valve (8) is arranged in a plane running at right angles to the longitudinal axis (3) of the container (2).

4. The inhalation arrangement in accordance with claim 1, characterised in that, the valve (8) has a dome or ball-shaped curvature in the direction of the longitudinal axis (3) of the container (2) and that at least one notch (22) is worked into the valve (8) which rises in the flow direction (11).

5. The inhalation arrangement in accordance with claim 4, characterised in that, one or more projections is/are provided in the area of the notches (22) by means of which the notches (22) are covered, and that the projections run above the adjacent notches (22) in relation to the flow direction (11) of the aerosol (6′).

6. The inhalation arrangement in accordance with claim 1, characterised in that, a positive aerosol pressure prevails in the first chamber (4) generated by the dosing device (6), that the level of preload on the valve (8) is such that the positive pressure prevailing in the first chamber (4) is enclosed therein and that a positive pressure generated in the second chamber (5) due to inhalation causes the valve (8) to be lifted in the direction of the second chamber (5).

7. The inhalation arrangement in accordance with claim 1, characterised in that, the container (2) is configured in two parts, that both parts of the container (2) are connected to one another in a releasable connection by means of screws, a thread (16) or a click process, and that the ring-shaped collar (21) attaches the valve (8) in the parting plane of both parts of the container (2) or it is fixed by other means, with the effect that the valve (8) and the ring-shaped collar (21) are secured.

8. The inhalation arrangement in accordance with claim 1, characterised in that, the end of the notch (22) runs flush with the longitudinal axis (3) of the container (2) during the inhalation and exhalation procedure.
